(19) United States
(12) Patent Application Publication
(10) Pub. No.: US 2007/0213681 A1
(43) Pub. Date: Sep. 13, 2007

(54) Method for the treatment of peyronie's disease

(75) Inventors: Ronald R. Manna, Valley Stream, NY (US); W. Paul Constantine, Centerport, NY (US)

Correspondence Address: COLEMAN SUDOL SAPONE, P.C., 714 COLORADO AVENUE, BRIDGE PORT, CT, 06605-1601, US

(73) Assignee: MISONIX, INCORPORATED, Farmingdale, NY (US)

(21) Appl. No.: 11/371,708

(22) Filed: Mar. 9, 2006

(51) Int. Cl. A61M 31/00

(52) U.S. Cl. 604/500

(57) ABSTRACT

In a method for treating Peyronie's disease comprises, one forms an incision in a patient's penis and inserts a distal end portion of an elongate probe through the incision so that a distal tip of the probe is in contact with a calcified lesion or fibrotic plaque in the penis. Thereafter the probe is vibrated to disrupt the calcified lesion or fibrotic plaque at a surgical site in the penis. The disrupted calcified lesion or fibrotic plaque is aspirated from the surgical site. Subsequently and after all instruments have been removed from the penis, the incision is closed.

### BACKGROUND OF INVENTION

This invention relates to a medical method for the treatment of penile disease. More particularly, this invention relates to a method for the treatment of Peyronie's disease.

There are many underlying reasons for male sexual dysfunction. Such causes as diabetes, Parkinson's disease, stroke, aging, atherosclerotic disease and side effects of drugs are common and well known. Such diseases may or may not be treated by secondary drugs or surgical techniques. One disease is less well known but causes troubling symptoms in males nonetheless. This is Peyronie's disease, a disorder of the connective tissue of the penis.

Peyronie's disease affects the connective tissue of the penis PN, specifically the tunica albuginea TA of the corpora cavemosa CC, shown in FIG. 1. As the disease progresses, fibrotic plaque FP (FIG. 2) forms in the tunica albuginea TA. This plaque FP grows and becomes noticeable beneath the skins and deforms the shaft. Because the plaque is inelastic and does not expand at the same rate as the balance of the penile tissue, upon erection the penis tends to bend or curve toward the side of plaque formation. Such curves may be complex and embarrassing. Rigidity of the penis is compromised and insertion becomes a problem. Pain may be associated with the erection as well. All of these symptoms cause consternation and embarrassment to the patient. Psychological impotence may occur even if physical impotence is not apparent. Although these symptoms are reversible in younger men, men over 50 show a lower spontaneous improvement rate and require medical intervention for relief.

Up until this point, several regimens have been employed to manage this disease. One is watchful waiting. This entails doing nothing but monitoring the progression of the disease in sexually inactive men. If the disease causes no additional pathology, it is generally left alone. If the disease is severe or more normal sexual performance is desired, then several interventions can be considered.

One intervention is drug therapy. Injections of steroids, Vitamin E or Protaba into the fibroids, are commonly used as a first pass treatment. It has been reported, though, that these drug based therapies are not clearly efficacious.

In cases where drugs are not viable or have failed, surgery is the only other option. Most medical references suggest a one year waiting period after diagnosis of Peyronie's disease before surgery is attempted, in order to provide time for spontaneous reversal of symptoms. After this time, a surgical grafting procedure is used where the lesions are replaced with skin from the inguinal crease. The lesions are excised and this skin placed in their stead. Care must be taken not to cut too deeply into the cavernosal tissue since damage may result in erectile dysfunction.

However, even with the best surgical techniques, it has been reported that in 75 to 84% of postoperative cases, erectile dysfunction is present. In these cases, penile implants are then required to provide rigidity for intercourse.

As can be imagined by this description of Peyronie's disease and its treatment, the patient is left with a Hobson's Choice: do nothing and remain disfigured and in pain or submit to multiple surgery with the resulting permanent scars and unnatural structures within the body rendering the penis permanently stiff.

Therefore, another option must be found to provide for immediate relief of Peyronie's disease without the need for invasive surgery and the concomitant high erectile dysfunction rate.

### OBJECT OF THIS INVENTION

It is a general object of the present invention to provide a method for the treatment of at least one penile disease.

A more particular object of this invention is to provide a method and/or medical instrumentation for the treatment of Peyronie's disease.

A more specific object of this invention is to provide a minimally invasive surgical means of treating Peyronie's disease.

It is another object of this invention to provide a method of use of surgical instrumentation in the treatment of Peyronie's disease.

These and other objects of the invention will be apparent from the drawings and descriptions herein. Although every object of the invention is attained in at least one embodiment of the invention, there is not necessarily any embodiment which attains all of the objects of the invention.

### SUMMARY OF THE INVENTION

The present invention provides a means of treating Peyronie's disease by use of ultrasound. Optionally, Peyronie's disease may be treated additionally or alternatively by Power Assisted Suction devices. In another option, Peyronie's disease may be treated additionally or alternatively by tissue suction.

A method for treating Peyronie's disease comprises, in accordance with the present invention, (a) forming an incision in a patient's penis, (b) inserting a distal end portion of an elongate probe through the incision so that a distal tip of the probe is in contact with a calcified lesion or fibrotic plaque in the penis, (c) thereafter vibrating the probe to disrupt the calcified lesion or fibrotic plaque at a surgical site in the penis, (d) aspirating the disrupted calcified lesion or fibrotic plaque from the penis, (e) removing all instruments from the penis, and (f) after the aspirating of the disrupted calcified lesion or fibrotic plaque from the penis and after the removing of all instruments, closing the incision.

The incision may be closed by any appropriate technique including suturing, stapling, gluing, taping, etc.

The aspirating of the disrupted material may be accomplished via a channel in the probe or via another instrument. The other instrument may be inserted into the penis through the one incision or through another incision.

The method may additionally comprise applying a liquid solution to the surgical site prior to or during the vibrating of the probe. The liquid solution may be a simple saline solution for assisting in the disruption or disintegration process. Alternatively, the liquid solution may include various medicaments or pharmaceutical agents such as coagulation agents, anesthetic compounds, etc.

The liquid solution may be applied by feeding the solution via a central or annular channel of the probe. Alternatively, a separate cannula may be used to convey the solution to the surgical site, either through the one incision prior to the insertion of the probe or through another incision. Where the liquid solution is applied via the probe, the aspiration may be effectuated through the same channel or through another channel provided in the probe.

The method may also comprise applying a cauterization current to the surgical site prior to the removing of all instruments from the penis. The electrical current may be applied via the probe or via another instrument inserted either through the one incision or through another incision in the skin of the penis. The electrical cauterization current may be applied via a monopolar circuit or a bipolar circuit.

The probe is either an ultrasound probe vibrated at ultrasonic frequencies or a hollow metal cannula vibrated at frequencies substantially below ultrasonic frequencies.

The method may further comprise moving, during the vibrating of the probe, the probe back and forth over the calcified lesion or fibrotic plaque in a plane substantially parallel to a skin surface of the penis.

### BRIEF DESCRIPTION OF THE DRAWINGS

FIG. 1 is a partial perspective view, partially in cross-section, of a normal human penis.

FIG. 2 is a partial perspective view, partially in cross-section, of a human penis wherein calcified lesion or fibrotic plaque has formed.

FIG. 3 is a partial perspective view, partially in cross-section, showing such a penis being treated with a distal end portion of a medical device, pursuant to the present invention.

FIG. 4 is a partial side elevational view of the penis and medical device of FIG. 3, on a larger scale.

FIG. 5 is a perspective view of medical instrumentation used to carry out the treatment of FIGS. 3 and 4.

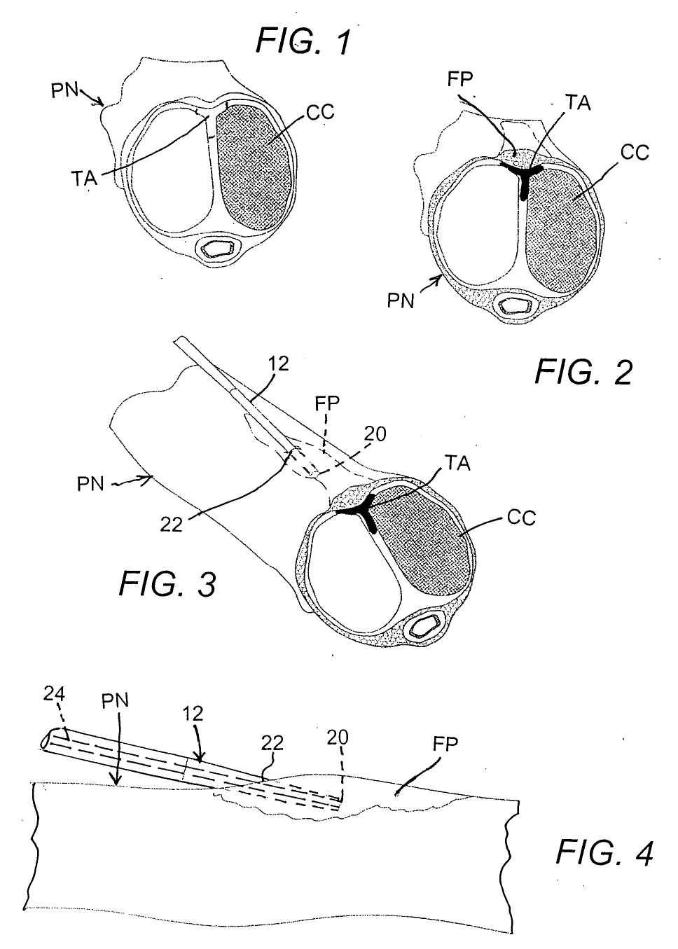

### DETAILED DESCRIPTION

Peyronie's disease is characterized by the formation of a calcified lesion or fibrotic plaque FP (FIG. 2) in the tunica albuginea TA of the penis PN, just below the skin level. As these formations grow, lumps develop and the penis looses its smooth appearance, the penis may bend during erection and pain may be present when the man is aroused. The only sure treatment is the removal of these lesions.

In order to remove the lesions with the least possible trauma, a method has been devised where the lesions are accessed in a minimally invasive scenario, disrupted internally and then aspirated from the penis with air suction. More particularly, as shown in FIG. 3 and 4, an ultrasound probe 12 is inserted below the skin of the penis PN and operated to disrupt the calcified lesion or fibrotic plaque FP.

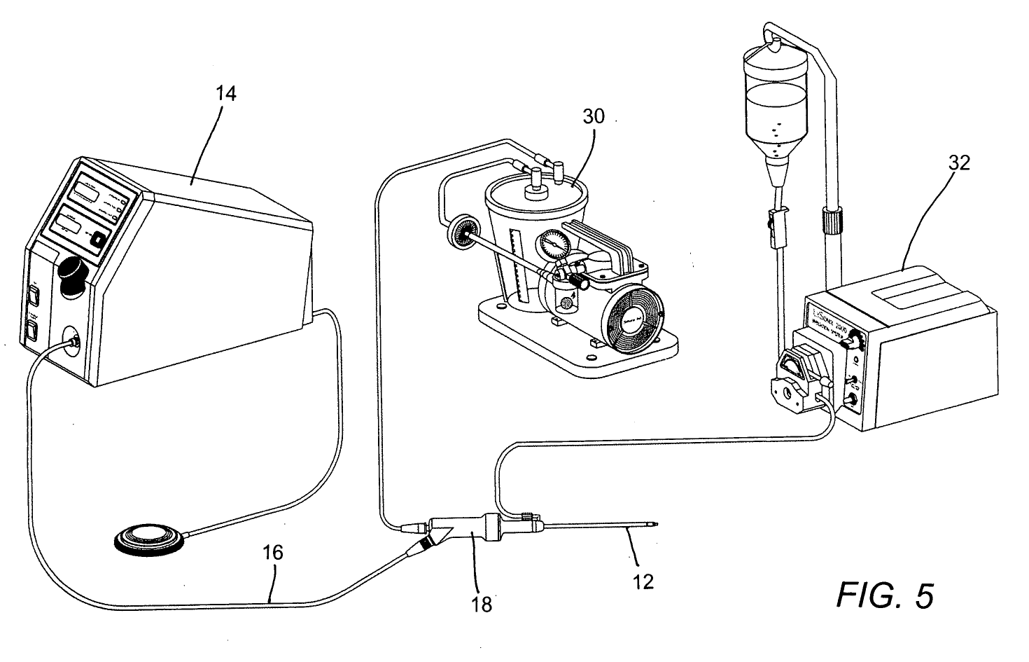

FIG. 5 shows the hardware or instrumentation necessary to accomplish this surgery. The hardware comprises an electronic generator 14, a handpiece cable 16, a piezoelectric or magnetostrictive handpiece 18, and tuned probe 12. Handpiece 18 and probe 12 are additionally connected to a vacuum pump 30 and an irrigation pump 32. The hardware of FIG. 5 is similar to the type needed for Suction Assisted Lipectomy (or Liposuction) procedures.

When the system of FIG. 5 is activated, generator 14 produces an output frequency in the ultrasound range between 16,000 and 150,000 Hz. The signal is sent to handpiece 18 via cable 16. Handpiece 18 is attached to probe 12 via a threaded joint or other connection known to the art. The piezoelectric crystals or magnetostrictive coil and stack of handpiece 18 convert the electrical signal to a mechanical vibration. This vibration is nominally the tuned resonant frequency of the handpiece/probe assembly. Probe 12 is designed to amplify the vibrations so that an excursion of between 5 and 500 microns is available at the tip. When a tip 20 of probe 12 is placed up against body tissue and the calcified lesion or fibrotic plaque FP, the tissue and lesion are ablated and liquefied. The liquefied material may then be aspirated from the surgical site with standard suction techniques.

Generator 14 incorporates known feedback circuitry to monitor and adjust its output so that the electrical signal tracks changes in the resonant frequency and power requirements of the system. Such devices have been described for many years and are well known to the art. Probe 12 and handpiece 18 may be cannulated (both elements provided with a central communicating bore) so that aspirant may be removed coaxially simultaneously during the procedure. Irrigants such as saline or water may be applied for cooling purposes or to increase the ablative effects of the ultrasound. Anesthetics such as lidocaine may be added to the irrigant fluid for pain suppression if desired. Vasocontrictive drugs such as epinephrine may be added to the irrigant to control topical bleeding.

In practice, the surgeon will prepare the patient with standard preoperative techniques. The calcified lesion or fibrotic plaque FP is located and mapped. An incision 22 (FIG. 3) is made in the skin of the penis PN which is not deeper than the level of the tunica albuginea TA so as not to injure that tissue. Ultrasound probe 12 is inserted through incision 22 and then the power turned on. The surgeon will move the probe 12 in and out in a piston stroke fashion, changing the direction of the probe tip 20 on each stroke in order to cover nearly the entire surface area of the lesion site. Probe 12 is typically moved in a plane that is substantially parallel to the skin surface and not directed down into the tunica albuginea TA.

The ultrasound vibration generated by probe tip 20 in the calcified lesion or fibrotic plaque FP breaks down and ablates the fibrous plaque lesions. This disintegrated matter may be aspirated from the surgical site by applying suction either via probe 12 or via a separate instrument. In the latter case, the solid ultrasound probe 12 is removed from the penis PN and a hollow suction cannula (not shown) inserted through incision 22. In the former case, probe 12 may be provided with a central channel 24 to which a vacuum generator or suction source (not shown) is connected at the proximal end of the probe.

In cases where cooling of the tissue is desired or where very tenacious fibroids are encountered, liquid may be applied to the surgical site through incision 22 before the procedure by use of an infiltration cannula (not shown). Conversely, the ultrasonic system may incorporate a liquid feed system to inject liquid through center channel 24, ultrasonically treat the lesion and then aspirate out the same channel.

When the calcified lesion or fibrotic plaque FP has been debulked sufficiently and the adhesions released, probe 12 is removed and incision 22 is sutured.

Certain cases may require debulking of the calcified lesion or fibrotic plaque FP without the use of ultrasound power. In these cases, Power Assisted Lipectomy devices may be employed in a similar manner as the ultrasound systems. Here a hollow metal cannula is inserted into the site. A power handpiece moves the cannula in and out at frequencies much lower than ultrasound, such as between 10 and 100 Hz or more. The excursions are generally much larger than those available with ultrasonic devices. However, this instrumentation does not have the ability to coagulate tissue bleeders as well nor ablate tissue. The main purpose is to allow the advance of the cannula more smoothly and with less overall trauma than a simple suction cannula used for suctioning out tissue. The method of use would generally be the same as that for ultrasound probe 12.

If severe bleeding is expected, the hardware may incorporate an RF coagulation feature wherein the probe is the monopole for the RF energy. The RF current would be engaged only upon the onset of uncontrolled bleeding from the surgery. A bipolar attachment may also be used where monopolar devices are not desired. Ultrasound probes with a cautery functionality are disclosed in U.S. Pat. Nos. 6,736,814 and 6,648,839, the disclosures of which are hereby incorporated by reference.

The word “probe” is used herein to broadly designate any medical instrument inserted into a penis for purposes of effectuating a surgical procedure at a surgical site in the penis. A probe may include multiple functionalities, such as the application of mechanical vibratory energy, the conduction of electrical current (probe equipped with monopolar or bipolar electrical circuit components), the application of suction, the delivery of irrigation fluid containing one or more therapeutic chemical components, etc. Thus, a probe as that term is used herein may be provided with one or more channels for aspiration and/or the delivery of a liquid. In the case of multiple channels, the probe may include a central axial channel and a peripheral cannula that serves to define an annular channel. A probe that includes a cauterization capability incorporates one (monopolar) or more (bipolar) conductors for delivering electrical current to a surgical site.

The word “disrupt” is used herein to broadly denote a fragmentation, disintegration, liquefaction, ablating or shaving of a calcified lesion or fibrotic plaque.

Although the invention has been described in terms of particular embodiments and applications, one of ordinary skill in the art, in light of this teaching, can generate additional embodiments and modifications without departing from the spirit of or exceeding the scope of the claimed invention. Accordingly, it is to be understood that the drawings and descriptions herein are proffered by way of example to facilitate comprehension of the invention and should not be construed to limit the scope thereof.

What is claimed is:

1. A method for treating Peyronie's disease, comprising:
forming an incision in a patient's penis;
inserting a distal end portion of an elongate probe through said incision so that a distal tip of said probe is in contact with a calcified lesion or fibrotic plaque in the penis;
thereafter vibrating said probe to disrupt said calcified lesion or fibrotic plaque at a surgical site in the penis;
aspirating the disrupted calcified lesion or fibrotic plaque from the penis;
removing all instruments from the penis; and
after the aspirating of the disrupted calcified lesion or fibrotic plaque from the penis and after the removing of all instruments, closing said incision.

2. The method defined in claim 1, further comprising applying a liquid solution to said surgical site prior to or during the vibrating of said probe.

3. The method defined in claim 2 wherein the applying of said liquid solution includes feeding said liquid solution via said probe.

4. The method defined in claim 1, further comprising applying a cauterization current to said surgical site prior to the removing of all instruments.

5. The method defined in claim 4 wherein the applying of cauterization current includes conducting said cauterization current to the surgical site via said probe.

6. The method defined in claim 1 wherein said probe is an ultrasound probe and the vibrating of said probe is at ultrasonic frequencies.

7. The method defined in claim 1 wherein said probe is a hollow metal cannula and the vibrating of said probe is at frequencies substantially below ultrasonic frequencies.

8. The method defined in claim 1 wherein the aspirating of the disrupted calcified lesion or fibrotic plaque includes applying suction to said surgical site via said probe.

9. The method defined in claim 1, further comprising moving, during the vibrating of said probe, said probe back and forth over the calcified lesion or fibrotic plaque in a plane substantially parallel to a skin surface of the penis.
